(19) United States
(12) Patent Application Publication
(10) Pub. No.: US 2012/0309990 A1
(43) Pub. Date: Dec. 6, 2012

(54) Processes for the Purification of Lubiprostone

(75) Inventors: Gamini Weeratunga, Ancaster (CA); Kiran Kumar Kothakonda, Brantford (CA); Alfredo Paul Ceccarelli, Brantford (CA); Bhaskar Reddy Guntoori, Brantford (CA); Fan Wang, Hamilton (CA)

(73) Assignee: APOTEX PHARMACHEM INC., Brantford, ON (CA)

(21) Appl. No.: 13/517,045

(22) PCT Filed: Dec. 17, 2010
(86) PCT No.: PCT/CA2010/001987

§ 371 Date: Aug. 16, 2012

Related U.S. Application Data
(60) Provisional application No. 61/287,918, filed on Dec. 18, 2009.

(51) Int. Cl. C07D 311/94; C09K 3/00

(52) U.S. Cl. 549/396; 252/182.12

(57) ABSTRACT

There is provided processes for purification of Lubiprostone by formation of amine salts. Also provided are compounds of the Lubiprostone amine salt. Also provided are compositions comprising Lubiprostone and amines.

### TECHNICAL FIELD

This invention relates to the field of synthetic chemistry for making Lubiprostone and in particular to the purification of Lubiprostone and amine salts of Lubiprostone.

### BACKGROUND

Lubiprostone (1) is an E1 type prostaglandin derivative. It is marketed in the United States as Amitiza® and is used for the treatment of idiopathic chronic constipation, irritable bowel syndrome and post operative ilues. The use of Lubiprostone softens the stool, increases motility, and promotes spontaneous bowel movements (SBM). Chemically, Lubiprostone is 7-[(1R,3R,6R,7R)-3-(1,1-difluoropentyl)-3-hydroxy-8-oxo-2-oxabicyclo[4.3.0]non-7-yl]heptanoic acid. It is reported to exist largely in the monocyclic form under hydrous conditions and in the bi-cyclic form under anhydrous conditions.

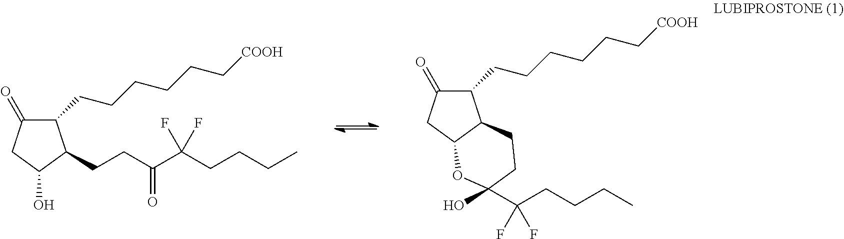

U.S. Pat. No. 5,117,042 discloses a method of treatment for improving encephalic function which comprises administering to a subject in need of such treatment a 15-keto-prostaglandin compound in an amount effective for improvement of encephalic function.

U.S. Pat. No. 5,284,858 discloses 13,14-dihydro-15-keto prostaglandins E having remarkable preventive effects against ulcers. Further, according to U.S. Pat. No. 5,284,858, 13,14-dihydro-15-ketoprostaglandins E have an advantage that they have none of side effects which prostaglandin E intrinsically has, or can remarkably reduce such effects of the prostaglandin E. According to U.S. Pat. No. 5,284,858, 13,14-dihydro-15-keto prostaglandins E are effective for animal and human use for treatment and prevention of ulcers, such as duodenal ulcer and gastric ulcer.

U.S. Pat. No. 7,355,064 discloses an improved method for preparing 15-keto prostaglandin E derivative. According to U.S. Pat. No. 7,355,064, the deprotection of protected hydroxyl group required in manufacturing a 15-keto-prostaglandin derivative is conducted under the presence of a phosphoric acid compound.

US 2007244333 discloses a method for preparing a prostaglandin derivative of formula (A):

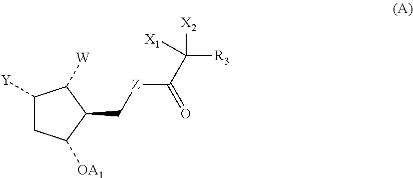

which comprises reacting an aldehyde represented by formula (B):

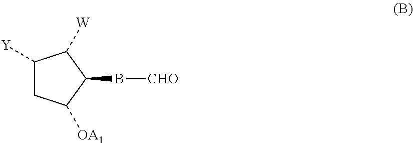

with a 2-oxoalkyl phosphonate in a reaction solvent in the presence of alkali hydroxide as sole base. According to US 2007244333, by carrying out the reaction using an alkali hydroxide as sole base in the reaction system, the desired prostaglandin derivative can be obtained by simple procedures and with high yield.

U.S. Pat. No. 5,229,529 provides a method of preparing alpha, beta—unsaturated ketolactones which are useful for production of prostaglandins having one or more halogen substituent(s) at the 16 or 17 portion in high yield, in which, a dimethyl (2-oxoalkyl) phosphonate having one or more halogen substituents, a starting material, is reacted with a bicyclolactone aldehyde under the presence of an alkali metal hydride and a zinc compound.

U.S. Pat. No. 5,468,880 discloses an improvement in the technique of synthesizing prostaglandins, particularly those having at least one halogen atom at the 16- or 17-position, which comprises introducing a omega chain into the aldehyde thereby to enable considerable yield improvement in the production of alpha, beta-unsaturated ketones, and which does not involve hydrogen generation and can insure safe operation. U.S. Pat. No. 5,468,880 discloses a method of producing alpha, beta-unsaturated ketones by reacting aldehyde with 2-oxoalkyl phosphonate, wherein the reaction was carried out under the presence of a base and a zinc compound.

U.S. Pat. No. 6,414,016 provides an anti-constipation composition containing a halogenated-bi-cyclic compound as an active ingredient in a ratio of bi-cyclic/mono-cyclic structure of at least 1:1. The halogenated-bi-cyclic compound in U.S. Pat. No. 6,414,016 is represented by Formula (I):

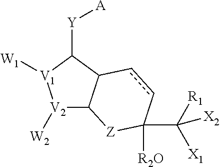

where X1 and X2 are preferably both fluorine atoms. According to U.S. Pat. No. 6,414,016, the composition can be used to treat constipation without substantive side-effects, such as stomachache.

WO2009121228 discloses a Lubiprostone crystal, its preparation process, its pharmaceutical composition or kit, and its use for the preparation of a pharmaceutical composition for treating gastroenteropathy, especially constipation. According to WO2009121228, the characteristic peaks of 20 reflection angle in X-ray powder diffraction spectra of the crystal include 14.6±0.2°, 17.0±0.2° and 19.6±0.2°. According to WO2009121228, the crystal has the advantages of high purity, stable property, and convenient storage and usage compared with amorphous Lubiprostone.

### SUMMARY

Lubiprostone is a difficult chemical to synthesize in a manner that provides for suitable purity for use in a pharmaceutical preparation. Many impurities often associated with Lubiprostone may be difficult to separate from Lubiprostone. This invention is based, at least in part, on the discovery of Lubiprostone t-butylamine salt. Forming a stable amine salt of Lubiprostone using t-butylamine allows impurities to be separated from Lubiprostone. Impurities that are removed by forming a t-butylamine salt of Lubiprostone include, but are not limited to, those impurities which are often associated with Lubiprostone and/or are difficult to separate from Lubiprostone. Additionally, t-butylamine is pharmaceutically suitable relative to other amines.

The present invention is based, at least in part, on processes for purifying Lubiprostone by forming an amine salt of the carboxylate moiety of Lubiprostone. Formation of an amine salt of the carboxylate moiety of Lubiprostone may reduce and/or eliminate the need for other purification techniques such as column chromatography and/or preparative high performance liquid chromatography. In illustrative embodiments of this invention, pharmaceutically acceptable class 3 solvents and inexpensive reagents may be used to produce pharmaceutically acceptable Lubiprostone. Purification processes of this type are amenable to commercial production of Lubiprostone in a cost effective and efficient manner.

Of particular interest are amine salts of Lubiprostone which form precipitates. Examples of such amine salts include, but are not limited to, Lubiprostone t-butylamine salt, Lubiprostone 1-phenylethylamine salt and Lubiprostone benzylamine salt. These salts result in selective precipitation of Lubiprostone and provide for efficient separation of Lubiprostone from impurities. Additionally, oils, as well as other viscous liquid forms of amine salts, also provide properties that aid in the separation of Lubiprostone from impurities.

Impurities that may be removed by forming an amine salt, for instance the t-butylamine salt of Lubiprostone, include, but are not limited to, stereoisomers of Lubiprostone (which has 3 chiral centres) and alkene-type by-products resulting from the elimination of the hydroxyl group on the cyclopentane ring.

Illustrative embodiments of the present invention provide Lubiprostone t-butylamine salt.

Illustrative embodiments of the present invention provide Lubiprostone t-butylamine salt having a PXRD diffractogram comprising peaks, in terms of degrees 2θ, at approximately 5.3, 7.7, 11.3, 16.0, 16.8, 17.2, 19.7 and 20.2.

Illustrative embodiments of the present invention provide Lubiprostone t-butylamine salt having a 1% KBr FTIR spectrum comprising peaks, in terms of cm⁻¹, at approximately 3226, 2935, 2883, 2218, 1749, 1543, 1526, and 1409.

Illustrative embodiments of the present invention provide Lubiprostone t-butylamine salt having a DSC thermogram comprising an endothermic peak with a peak onset temperature of approximately 93° C. and a peak maximum of approximately 97° C.

Illustrative embodiments of the present invention provide Lubiprostone t-butylamine salt having a PXRD diffractogram substantially similar to a PXRD diffractogram as depicted in FIG. 1.

Illustrative embodiments of the present invention provide Lubiprostone t-butylamine salt having a FTIR spectrum substantially similar to a FTIR spectrum as depicted in FIG. 2.

Illustrative embodiments of the present invention provide Lubiprostone t-butylamine salt having a DSC thermogram substantially similar to a DSC thermogram as depicted in FIG. 3.

Illustrative embodiments of the present invention provide a process to prepare Lubiprostone comprising: forming a solution of Lubiprostone in a first organic solvent; adding an amine to the solution of Lubiprostone in the first organic solvent thereby forming a Lubiprostone amine salt; and isolating the Lubiprostone amine salt.

Illustrative embodiments of the present invention provide a process described herein further comprising regenerating the Lubiprostone free acid by adjusting the pH.

Illustrative embodiments of the present invention provide a process described herein further comprising extracting the Lubiprostone free acid into a second organic solvent.

Illustrative embodiments of the present invention provide a process described herein further comprising crystallizing the Lubiprostone free acid.

Illustrative embodiments of the present invention provide a process described herein further comprising purifying the isolated Lubiprostone amine salt.

Illustrative embodiments of the present invention provide a process described herein further comprising regenerating the Lubiprostone free acid by adjusting the pH.

Illustrative embodiments of the present invention provide a process described herein further comprising extracting the Lubiprostone free acid into a second organic solvent.

Illustrative embodiments of the present invention provide a process described herein further comprising crystallizing the Lubiprostone free acid.

Illustrative embodiments of the present invention provide a process described herein wherein first organic solvent is selected from the group consisting of: C₄ to C₉ alkyl esters, C₄ to C₈ alkyl ethers and mixtures thereof.

Illustrative embodiments of the present invention provide a process described herein wherein the first organic solvent is selected from the group consisting of: ethyl acetate, methyl t-butyl ether and mixtures thereof.

Illustrative embodiments of the present invention provide a process described herein wherein a volume of first organic solvent is between about 1 volume to about 15 volumes.

Illustrative embodiments of the present invention provide a process described herein wherein the amine is t-butylamine.

Illustrative embodiments of the present invention provide a process described herein wherein an equivalent of amine is between about 0.95 equivalents to about 1.05 equivalents.

Illustrative embodiments of the present invention provide a process described herein wherein the pH is adjusted to between about pH 4.5 to about pH 6.5.

Illustrative embodiments of the present invention provide a process described herein wherein the pH is adjusted using between about 1.0 equivalents to about 1.1 equivalents of formic acid.

Illustrative embodiments of the present invention provide a process described herein wherein the second organic solvent is a C₄ to C₉ alkyl ester.

Illustrative embodiments of the present invention provide a process described herein wherein the second organic solvent is a C₄ to C₉ alkyl ester and the crystallizing comprises using a C₅ to C₁₀ hydrocarbon as an antisolvent.

Illustrative embodiments of the present invention provide a process described herein wherein a volume of second organic solvent to antisolvent is about 1:40 (vol:vol) to about 1:6 (vol:vol).

Illustrative embodiments of the present invention provide a process described herein wherein the second organic solvent is ethyl acetate.

Illustrative embodiments of the present invention provide a process described herein wherein the second organic solvent is ethyl acetate and the crystallizing comprises using petroleum ether as an antisolvent.

Illustrative embodiments of the present invention provide a process described herein wherein a volume of second organic solvent to antisolvent is about 1:40 (vol:vol) to about 1:6 (vol:vol).

Illustrative embodiments of the present invention provide a process described herein wherein the forming the Lubiprostone amine salt comprising precipitation.

Illustrative embodiments of the present invention provide a Lubiprostone amine salt of formula (I):

Lubiprostone.NR¹R²R³ (I)

wherein R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl; or two of R¹, R² and R³ together with the nitrogen to which they are bonded form a single C₄-C₈ ring group and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl.

Illustrative embodiments of the present invention provide a Lubiprostone amine salt described herein wherein R¹ and R² together with the nitrogen to which they are bonded form a single C₄-C₈ ring group and the ring group contains an additional heteroatom.

Illustrative embodiments of the present invention provide a Lubiprostone amine salt described herein wherein the additional heteroatom is a nitrogen or an oxygen.

Illustrative embodiments of the present invention provide a composition comprising Lubiprostone and an amine of formula (II):

NR¹R²R³ (II)

wherein R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl; or two of R¹, R² and R³ together with the nitrogen to which they are bonded form a single C₄-C₈ ring group and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl.

Illustrative embodiments of the present invention provide a composition described herein wherein R¹ and R² together with the nitrogen to which they are bonded form a single ring group and the ring group contains an additional heteroatom.

Illustrative embodiments of the present invention provide a composition described herein wherein the additional heteroatom is a nitrogen or an oxygen.

Illustrative embodiments of the present invention provide a composition comprising Lubiprostone and t-butylamine.

Illustrative embodiments of the present invention provide a composition comprising Lubiprostone and methyl tert-butyl ether.

Illustrative embodiments of the present invention provide Lubiprostone 1-phenethylamine salt.

Illustrative embodiments of the present invention provide Lubiprostone benzylamine salt.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Drawings which illustrate embodiments of the invention are:

FIG. 1: is a PXRD diffractogram of Lubiprostone t-butylamine salt.

FIG. 2: is an IR spectrum of Lubiprostone t-butylamine salt.

FIG. 3: is a DSC thermogram of Lubiprostone t-butylamine salt.

### DETAILED DESCRIPTION

When used in reference to a diffractogram, a spectrum and/or data presented in a graph, the term “substantially similar” means that the subject diffractogram, spectrum and/or data presented in a graph encompasses all diffractograms, spectra and/or data presented in graphs that vary within acceptable boundaries of experimentation that are known to a person of skill in the art. Such boundaries of experimentation will vary depending on the type of the subject diffractogram, spectrum and/or data presented in a graph, but will nevertheless be known to a person of skill in the art.

When used in reference to a peak in a PXRD diffractogram, the term “approximately” means that the peak may vary by ±0.2 degrees 2θ of the subject value.

When used in reference to a peak in a FTIR spectrum, the term “approximately” means that the peak may vary by ±5 cm⁻¹ of the subject value.

When used in reference to a peak in a DSC thermogram, the term “approximately” means that the peak may vary by ±1 degree of the subject value.

As used herein when referring to a diffractogram, spectrum and/or to data presented in a graph, the term “peak” refers to a feature that one skilled in the art would recognize as not attributing to background noise.

Depending on the nature of the methodology applied and the scale selected to display results obtained from an X-ray diffraction analysis, an intensity of a peak obtained may vary quite dramatically. For example, it is possible to obtain a relative peak intensity of 0.01% when analyzing one sample of a substance, but another sample of the same substance may show a much different relative intensity for a peak at the same position. This may be due, in part, to the preferred orientation of the sample and its deviation from the ideal random sample orientation, sample preparation and the methodology applied. Such variations are known and understood by a person of skill in the art.

As used herein, the term “substituted” refers to the replacement of a hydrogen atom on a compound with a substituent group. A substituent may be a non-hydrogen atom or multiple atoms of which at least one is a non-hydrogen atom and one or more may or may not be hydrogen atoms.

As used herein, the term “alkyl” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (e.g. C1-C10 or 1- to 10-membered means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “lower alkyl” comprises straight chain or branched chain saturated hydrocarbon groups having 1 to 6 carbon atoms, for instance, methyl, ethyl, propyl, isopropyl, butyl, isobutyl, sec-butyl, and t-butyl. Lower alkyls may be substituted or unsubstituted.

The term “short chain alkyl” means an alkyl group having 1 to 4 carbon atoms. Short chain alkyls may be substituted or unsubstituted.

As used herein, the term “aryl” by itself or as part of another substituent, means, unless otherwise stated, a polyunsaturated, aromatic, hydrocarbon substituent which can be a single ring or multiple rings (often from 1 to 3 rings) which are fused together or linked covalently. “Aryl” includes, but is not limited to, “heteroaryl” groups. “Heteroaryl” refers to an aryl group that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include: phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridinyl, 3-pyridinyl, 4-pyridinyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, etc.) including those alkyl groups in which a carbon atom containing group (e.g., a methylene group) has been replaced by, for example, an oxygen atom (e.g., phenoxymethyl, 2-pyridyloxymethyl, 3-(1-naphthyloxy)propyl, etc).

Crude Lubiprostone may be prepared by methods known in the art, including but not limited to methods described in U.S. Pat. No. 5,117,042, and U.S. Pat. No. 7,355,064.

According to the illustrative embodiments of the present invention, pharmaceutically acceptable Lubiprostone may be prepared from an amine salt of general formula Lubiprostone.NR¹R²R³ wherein R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl. Alternatively, two of R¹, R² and R³ together with the nitrogen to which they are bonded may form a single C₄-C₈ ring group with or without an additional heteroatom and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl. If an additional heteroatom is present in such a ring group, the heteroatom is often, but not always, nitrogen or oxygen.

Crude Lubiprostone may be purified by forming an amine salt, purifying the amine salt and forming Lubiprostone free acid. Optionally, this may be followed by crystallization of the Lubiprostone free acid.

In an illustrative embodiment, the present invention comprises a process for the preparation of an amine salt of Lubiprostone comprising:
a. dissolving Lubiprostone in an organic solvent or a mixture of organic solvents at ambient temperature thereby forming a Lubiprostone solution;
b. adding to the solution an amine of general formula NR¹R²R³:
   wherein R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl; or
      two of R¹, R² and R³ together with the nitrogen to which they are bonded form a single C₄-C₈ ring group and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl,
   thereby forming the amine salt as a solid; and
c. isolating the amine salt.

Optionally the process may further comprise:
d. purifying the amine salt.

The Lubiprostone may be dissolved in any organic solvent. The organic solvent may be a C₄ to C₉ ester, for example but not limited to, ethyl acetate. The organic solvent may be a C₄ to C₈ alkyl ether, for example but not limited to methyl t-butyl ether (MTBE). Often the solvent is ethyl acetate, MTBE or a mixture thereof. The volume of organic solvent may be from about 1 volume to about 15 volumes. The volume of organic solvent may be about 5 volumes to about 13 volumes.

An amount of amine that may be added to the Lubiprostone solution may be from about 0.5 equivalents to about 1.5 equivalents. Often the amount of amine that may be added to the Lubiprostone is about 0.95 equivalents to about 1.05 equivalents.

The Lubiprostone amine salt may be isolated by filtration. If desired, the salt may be purified further by processing the salt using a second solvent system having the same properties as the solvent system used to obtain the salt in the first place.

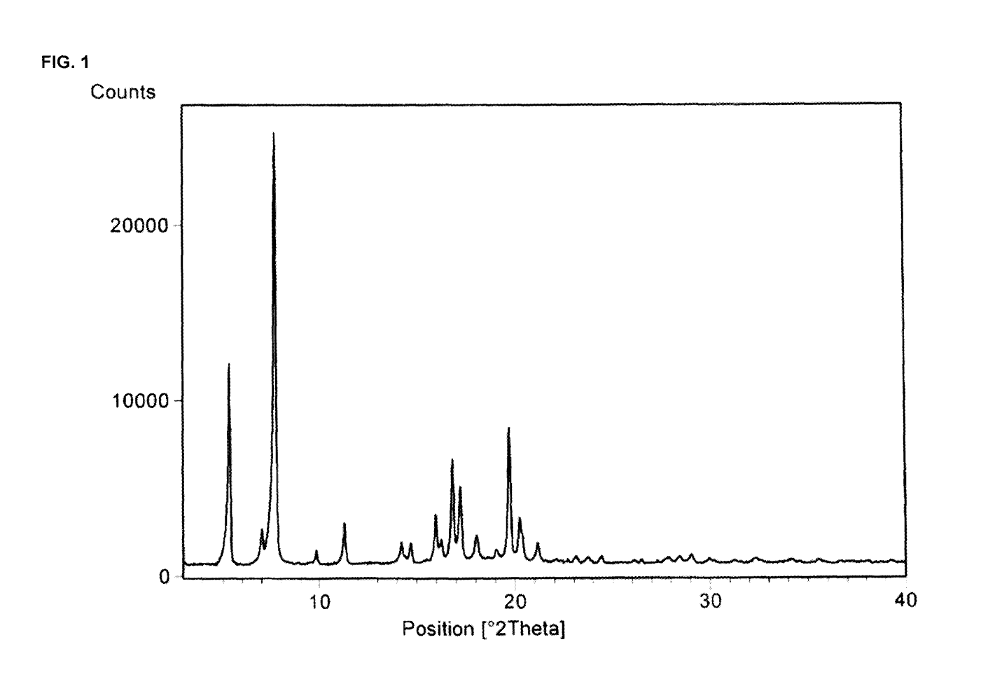

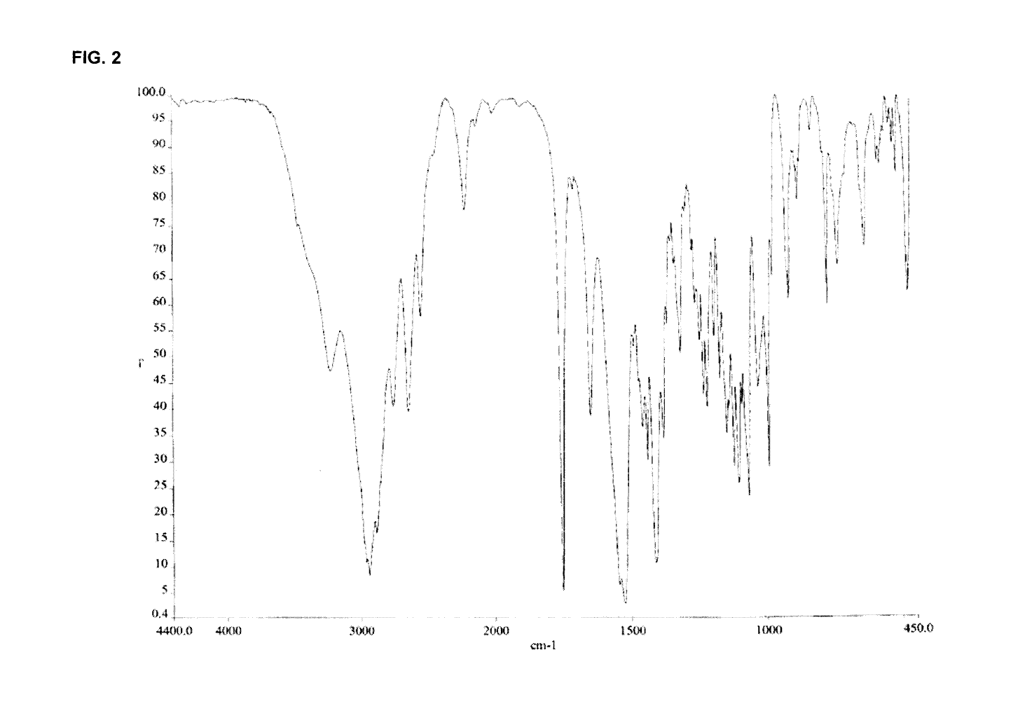

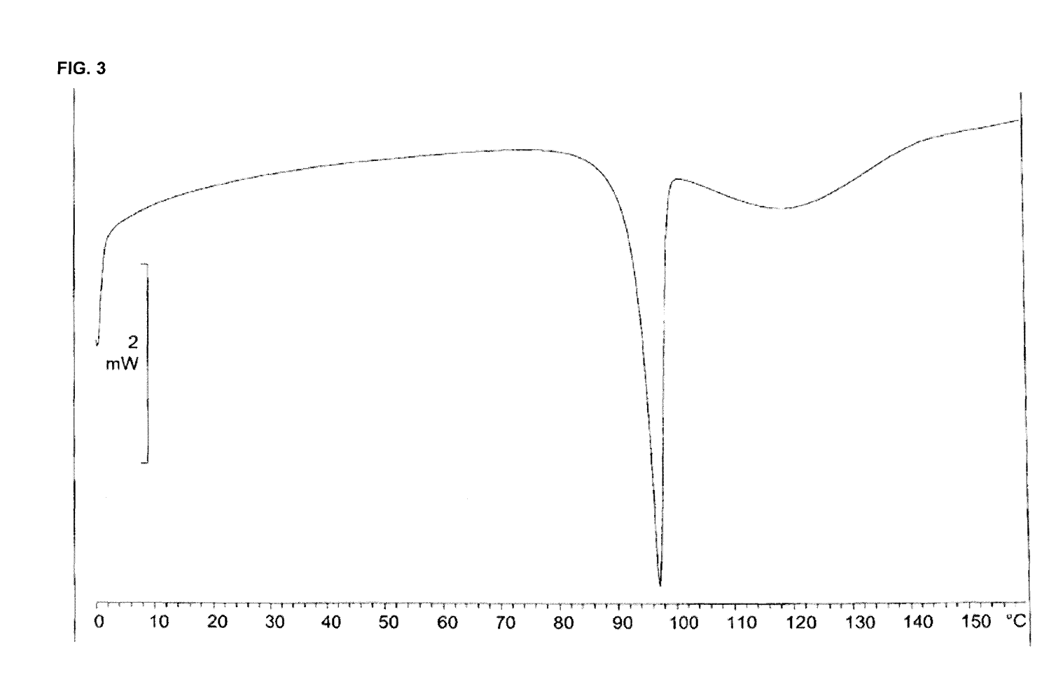

In an illustrative embodiment, the present invention comprises a form of Lubiprostone t-butylamine salt which is referred to herein as Form APO. An illustrative PXRD diffractogram of Form APO is given in FIG. 1. An illustrative IR spectrum of Form APO is given in FIG. 2. An illustrative DSC thermogram of Form APO is given in FIG. 3.

In another embodiment, the present invention provides a process for preparing pharmaceutically acceptable Lubiprostone comprising:
a. suspending Lubiprostone amine salt in a first organic solvent;
b. forming Lubiprostone free acid by adding an acid;
c. extracting the Lubiprostone free acid into a second organic solvent; and
d. isolating Lubiprostone.

The process may optionally further comprise crystallizing the Lubiprostone.

The first organic solvent used to suspend the Lubiprostone amine salt may be any organic solvent. Examples of suitable first organic solvents include, but are not limited to, C₄ to C₉ alkyl esters, such as ethyl acetate and C₄ to C₈ alkyl ethers, such as MTBE, a mixture thereof or a mixture of a C₄ to C₉ alkyl esters and C₅ to C₁₀ hydrocarbons such as petroleum ether.

The acid used to form the Lubiprostone free acid may be an organic acid. The acid may be formic acid in water. An amount of acid used may be from about 0.5 equivalents to about 1.5 equivalents. Often the amount of acid used is about 0.8 equivalents to about 1.2 equivalents. In other embodiments, the amount of acid used is about 1.0 equivalent to about 1.1 equivalents.

The pH of the Lubiprostone free acid solution can be from pH 4.5 to pH 6.5.

The second organic solvent used to isolate pharmaceutically acceptable Lubiprostone may be the same as the first organic solvent. Often the second organic solvent is ethyl acetate, petroleum ether or a mixture thereof. A ratio of ethyl acetate to petroleum ether may be from about 1:40 (vol:vol) to about 1:6 (vol:vol).

In another illustrative embodiment, the present invention provides a process of preparing pharmaceutically acceptable Lubiprostone from Lubiprostone t-butylamine salt comprising:

a. suspending Lubiprostone t-butylamine salt in a first organic solvent;

b. forming Lubiprostone free acid by adding an acid;

c. extracting the Lubiprostone free acid into a second organic solvent; and

d. isolating Lubiprostone.

The process may optionally further comprise crystallizing Lubiprostone.

The first organic solvent used to suspend the Lubiprostone t-butylamine salt may be any organic solvent. Often the first organic solvent is a C₄ to C₉ alkyl esters, such as ethyl acetate, a C₄ to C₈ alkyl ether, such as MTBE, a mixture thereof, or a mixture of a C₄ to C₉ alkyl ester and a C₅ to C₁₀ hydrocarbons. An example of a C₅ to C₁₀ hydrocarbon is petroleum ether. In some embodiments, the isolated Lubiprostone contains pharmaceutically acceptable levels of residual t-butylamine and solvents.

The following examples are illustrative of some of the embodiments of the invention described herein. These examples do not limit the spirit or scope of the invention in anyway.

### EXAMPLES

Powder X-Ray Diffraction Analysis: The data were acquired on a PANanalytical X-Pert Pro MPD diffractometer with fixed divergence slits and an X-Celerator RTMS detector. The diffractometer was configured in Bragg-Brentano geometry; data was collected over a 2 theta range of 3 to 40 using CuKα radiation at a power of 40 mA and 45 kV. CuKβ radiation was removed using a divergent beam nickel filter. A step size of 0.017 degrees was used. A step time of 200 seconds was used. Samples were rotated at 1 Hz to reduce preferred orientation effects. The samples were prepared by dusting a small amount of powder onto a lightly greased zero background holder. The resulting diffractogram was baseline subtracted.

Fourier Transform Infrared (FTIR) Analysis: The FTIR spectrum was collected at 4 cm⁻¹ resolution using a Perkin Elmer Paragon 1100 single beam FTIR instrument. The samples were intimately mixed in an approximately 1:100 ratio (w/w) with potassium bromide using an agate mortar and pestle to a fine consistency; the mixture was compressed in a pellet die at a pressure of 4 to 6 tonnes for a period of time between 2 and 5 minutes. The resulting disk was scanned 4 times versus a collected background. Data was baseline corrected and normalized.

Differential Scanning calorimetry (DSC) Analysis: The DSC thermograms were collected on a Mettler-Toledo 821e instrument. Samples (1 to 5 mg) were weighed into a 40 μL aluminum pan and were crimped closed with an aluminum lid. The samples were analyzed under a flow of nitrogen (ca. 55 mL/min) at a scan rate of 10° C./minute.

### Example 1

To a solution of crude Lubiprostone (10 g) having a purity by HPLC of 70.4% in ethyl acetate (10 vol) was added t-butylamine (1.05 eq) at room temperature. The reaction mixture was stirred at room temperature until precipitation of the amine salt occurred. The amine salt was isolated by filtration and dried to give Lubiprostone t-butylamine salt as depicted in the PXRD diffractogram in FIG. 1, the FTIR spectrum in FIG. 2 and the DSC thermogram in FIG. 3.

¹H NMR (CDCl₃): δ 0.91-0.96 (t, 3H, J=7.2 Hz), 1.19-1.74 (m, 26H), 1.78-2.01 (m, 7H), 2.12-2.36 (m, 3H), 2.52-2.61 (dd, 1H, J=17.6, 7.2 Hz), 4.13-4.22 (m, 1H), 6.46 (br s, 3H).

The amine salt was suspended in ethyl acetate (6 vol) and water (3 vol). The resulting bi-phasic mixture was adjusted to pH 5 with formic acid. The organic layer was separated and concentrated to obtain pure material as a syrup. Upon crystallization using ethyl acetate/petroleum ether (1:9 volumes), the syrup produced Lubiprostone in approximately 70% recovery and having a HPLC purity of 99.95%.

### Example 2

To a solution of crude Lubiprostone (3 g) in ethyl acetate (10 vol) was added t-butylamine (1.05 eq) at room temperature. The reaction mixture was allowed to stir at room temperature until precipitation of the amine salt occurred. The amine salt was isolated by filtration and then suspended in ethyl acetate/petroleum ether (6 vol, 3:1 v/v) and water (3 vol). The resulting bi-phasic mixture was adjusted to pH 5 with formic acid. The organic layer was separated and concentrated to furnish pure Lubiprostone as syrup. This was recrystallized using ethyl acetate/petroleum ether (1:9 volumes) to afford Lubiprostone crystals in 50% yield.

### Example 3

To a solution of crude Lubiprostone (1 g) in MTBE (10 vol) was added t-butylamine (1.05 eq) at room temperature. The reaction mixture was allowed to stir at room temperature until precipitation of the amine salt occurred. The amine salt was isolated by filtration and then suspended in ethyl acetate (6 vol) and water (3 vol). The resulting bi-phasic mixture was adjusted to pH 5 with formic acid. The organic layer was separated and concentrated to provide pure material as a syrup. Upon using ethyl acetate/petroleum ether (1:9 volumes), the corresponding syrup produced Lubiprostone crystals. Yield=55.1%.

### Example 4

To a solution of crude Lubiprostone (2 g) in MTBE (10 vol) was added t-butylamine (1.05 eq) at room temperature followed by petroleum ether (3 vol). The reaction mixture was allowed to stir at room temperature until precipitation of the amine salt occurred whereupon it was isolated by filtration. The amine salt was suspended in ethyl acetate (6 vol) and water (3 vol). The resulting bi-phasic mixture was adjusted to pH 5 with formic acid and the organic layer was separated and concentrated to obtain pure material as a syrup. Upon using ethyl acetate/petroleum ether (1:9 volumes), the corresponding syrup produced Lubiprostone crystals. Yield=60%.

### Example 5

To a solution of crude Lubiprostone (0.33 g) in MTBE (6 vol) was added 1-phenethylamine (1 eq) at room temperature, followed by additional MTBE (12 vol). The reaction mixture was stirred at room temperature until precipitation of the amine salt occurred. The amine salt was isolated by filtration, washed with MTBE and dried to give Lubiprostone 1-phenethylamine salt in approximately 70% recovery.

¹H NMR (CDCl₃): δ 0.91-0.96 (t, 3H, 7.3 Hz), 1.19-1.73 (m, 18H), 1.77-2.06 (m, 7H), 2.15-2.29 (m, 3H), 2.52-2.60 (dd, 1H, J=17.6, 7.2 Hz), 4.12-4.21 (m, 2H), 5.03 (br s, 4H), 7.28-7.35 (m, 5H).

### Example 6

To a solution of crude Lubiprostone (0.3 g) in MTBE (6 vol) was added benzylamine (1 eq) at room temperature, followed by additional MTBE (12 vol). The reaction mixture was stirred at room temperature until precipitation of the amine salt occurred. The amine salt was isolated by filtration, washed with MTBE and dried to give Lubiprostone benzylamine salt in approximately 60% recovery.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. Lubiprostone t-butylamine salt.

2. The Lubiprostone t-butylamine salt of claim 1 having a PXRD diffractogram comprising peaks, in terms of degrees 2θ, at approximately 5.3, 7.7, 11.3, 16.0, 16.8, 17.2, 19.7 and 20.2.

3. The Lubiprostone t-butylamine salt of claim 2 having a 1% KBr FTIR spectrum comprising peaks, in terms of cm⁻¹, at approximately 3226, 2935, 2883, 2218, 1749, 1543, 1526, and 1409.

4. The Lubiprostone t-butylamine salt of claim 2 having a DSC thermogram comprising an endothermic peak with a peak onset temperature of approximately 93° C. and a peak maximum of approximately 97° C.

5. The Lubiprostone t-butylamine salt of claim 1 having a PXRD diffractogram substantially similar to a PXRD diffractogram as depicted in FIG. 1.

6. The Lubiprostone t-butylamine salt of claim 5 having a FTIR spectrum substantially similar to a FTIR spectrum as depicted in FIG. 2.

7. The Lubiprostone t-butylamine salt of claim 5 having a DSC thermogram substantially similar to a DSC thermogram as depicted in FIG. 3.

8. A process to prepare Lubiprostone comprising:
forming a solution of Lubiprostone in a first organic solvent;
adding an amine to the solution of Lubiprostone in the first organic solvent thereby forming a Lubiprostone amine salt; and
isolating the Lubiprostone amine salt.

9. The process of claim 8 further comprising regenerating the Lubiprostone free acid by adjusting the pH.

10. The process of claim 9 further comprising extracting the Lubiprostone free acid into a second organic solvent.

11. The process of claim 10 further comprising crystallizing the Lubiprostone free acid.

12. The process of claim 8 further comprising purifying the isolated Lubiprostone amine salt.

13. The process of claim 12 further comprising regenerating the Lubiprostone free acid by adjusting the pH.

14. The process of claim 13 further comprising extracting the Lubiprostone free acid into a second organic solvent.

15. The process of claim 14 further comprising crystallizing the Lubiprostone free acid.

16. The process of claim 8 wherein first organic solvent is selected from the group consisting of: C₄ to C₉ alkyl esters, C₄ to C₈ alkyl ethers and mixtures thereof.

17. The process of claim 8 wherein the first organic solvent is selected from the group consisting of: ethyl acetate, methyl t-butyl ether and mixtures thereof.

18. The process of claim 8 wherein a volume of first organic solvent is between about 1 volume to about 15 volumes.

19. The process of claim 8 wherein the amine is t-butylamine.

20. The process of claim 8 wherein an equivalent of amine is between about 0.95 equivalents to about 1.05 equivalents.

21. The process of claim 13 wherein the pH is adjusted to between about pH 4.5 to about pH 6.5.

22. The process of claim 13 wherein the pH is adjusted using between about 1.0 equivalents to about 1.1 equivalents of formic acid.

23. The process of claim 14 wherein the second organic solvent is a C₄ to C₉ alkyl ester.

24. The process of claim 15 wherein the second organic solvent is a C₄ to C₉ alkyl ester and the crystallizing comprises using a C₅ to C₁₀ hydrocarbon as an antisolvent.

25. The process according to claim 24 wherein a volume of second organic solvent to antisolvent is about 1:40 (vol:vol) to about 1:6 (vol:vol).

26. The process of claim 14 wherein the second organic solvent is ethyl acetate.

27. The process of claim 15 wherein the second organic solvent is ethyl acetate and the crystallizing comprises using petroleum ether as an antisolvent.

28. The process according to claim 27 wherein a volume of second organic solvent to antisolvent is about 1:40 (vol:vol) to about 1:6 (vol:vol).

29. The process of claim 8 wherein the forming the Lubiprostone amine salt comprising precipitation.

30. A Lubiprostone amine salt of formula (I):
Lubiprostone.NR¹R²R³ (I)
wherein
R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl; or
two of R¹, R² and R³ together with the nitrogen to which they are bonded form a single C₄-C₈ ring group and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl.

31. The Lubiprostone amine salt of claim 30 wherein R¹ and R² together with the nitrogen to which they are bonded form a single C₄-C₈ ring group and the ring group contains an additional heteroatom.

32. The Lubiprostone amine salt of claim 31 wherein the additional heteroatom is a nitrogen or an oxygen.

33. A composition comprising Lubiprostone and an amine of formula (II):
NR¹R²R³ (II)
wherein
R¹, R² and R³ are each independently selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl; or
two of R¹, R² and R³ together with the nitrogen to which they are bonded form a single C₄-C₈ ring group and the R¹, R² or R³ group that is not part of the ring group is selected from the group consisting of: H, C₁-C₁₂ alkyl, substituted C₁-C₁₂ alkyl, C₃-C₁₂ aryl, substituted C₃-C₁₂ aryl, C₃-C₁₂ arylalkyl and substituted C₃-C₁₂ arylalkyl.

34. The composition of claim 33 wherein R¹ and R² together with the nitrogen to which they are bonded form a single ring group and the ring group contains an additional heteroatom.

35. The composition of claim 34 wherein the additional heteroatom is a nitrogen or an oxygen.

36. A composition comprising Lubiprostone and t-butylamine.

37. A composition comprising Lubiprostone and methyl tert-butyl ether.

38. Lubiprostone 1-phenethylamine salt.

39. Lubiprostone benzylamine salt.
